(19) United States
(12) Patent Application Publication
(10) Pub. No.: US 2024/0404652 A1
(43) Pub. Date: Dec. 5, 2024

(54) ENSEMBLE LEARNING-BASED OPTIMIZED EVALUATION METHOD FOR IMPROVEMENT EFFECT OF ORGANIC MATERIALS ON SOIL QUALITY

(71) Applicant: Institute of Environment and Sustainable Development in Agriculture, CAAS, Haidian District (CN)

(72) Inventors: Qingwen ZHANG, Haidian District (CN); Chang SHI, Haidian District (CN); Xiaoying ZHAN, Haidian District (CN); Zhuo HAO, Haidian District (CN)

(73) Assignee: Institute of Environment and Sustainable Development in Agriculture, CAAS, Haidian District (CN)

(21) Appl. No.: 18/653,172

(22) Filed: May 2, 2024

(30) Foreign Application Priority Data
Jun. 3, 2023 (CN) 202310650388.9

(51) Int. Cl. G16C 20/70; G01N 33/24

(52) U.S. Cl. CPC G16C 20/70; G01N 33/24

(57) ABSTRACT

Provided relates to the field of evaluation methods, and in particular, to an ensemble learning-based optimized evaluation method for an improvement effect of organic materials on soil quality, including the following steps: S1: formulating an overall framework; S2: establishing of a total dataset (TDS) and a minimum dataset (MDS), and calculating soil quality indices based on the TDS and the MDS; S3: developing a soil quality prediction model based on machine learning; S4: generating a soil quality extended dataset and a soil quality evaluation dataset; and S5: data analysis method. In step S1, the overall framework includes: establishment of the TDS and calculation of a soil quality index based on the TDS, establishment of the MDS and calculation of a soil quality index based on the MDS, development of the soil quality prediction model based on machine learning, and generation of the soil quality evaluation dataset.

### CROSS REFERENCE TO RELATED APPLICATION

This patent application claims the benefit and priority of Chinese Patent Application No. 202310650388.9 filed with the China National Intellectual Property Administration on Jun. 3, 2023, the disclosure of which is incorporated by reference herein in its entirety as part of the present application.

### TECHNICAL FIELD

The present disclosure relates to the field of evaluation methods, and in particular, to an ensemble learning-based optimized evaluation method for an improvement effect of organic materials on soil quality.

### BACKGROUND

Organic materials, as important soil amendments, can increase soil organic matter (SOM) content, improve soil structure, and enhance soil fertility and water retention capacity, thereby promoting the growth and development of crops while also contributing to environmental protection and reducing land degradation. With the development of organic agriculture and the increasing awareness of ecological environmental protection, the application of organic materials in soil improvement has garnered increasing attention. Different organic materials have varying chemical compositions and characteristics, leading to diverse impacts on soil quality. Animal-source organic materials such as organic fertilizers and manure provide nutrients and microorganisms, to promote soil biological activity and organic matter accumulation. Plant-source organic materials like straw and green manure improve soil structure and water retention capacity, thus fostering the development of soil aeration and biodiversity. Biochar enhances the carbon storage capacity of soil, optimizes soil pH and ion exchange capacity, and contributes to increasing soil fertility and water retention capability.

Therefore, developing a high-precision soil quality quantitative prediction model is crucial for revealing response rules of organic materials and soil quality under typical planting patterns.

### SUMMARY

To address shortcomings of prior art, it is crucial to develop a high-precision soil quality quantitative prediction model to reveal the response rules of organic materials and soil quality under typical planting patterns.

The present disclosure provides an ensemble learning-based optimized evaluation method for an improvement effect of organic materials on soil quality, including following steps:
S1: formulating an overall framework;
S2: establishing a total dataset (TDS) and a minimum dataset (MDS), and calculating soil quality indices based on the TDS and the MDS;
S3: developing a soil quality prediction model based on machine learning;
S4: generating a soil quality extended dataset and a soil quality evaluation dataset; and
S5: analyzing data in the soil quality extended dataset and the soil quality evaluation dataset by using the soil quality prediction model, to evaluate improvement effect of organic materials on soil quality.

In the embodiment, in step S1, the overall framework includes four aspects: establishment of the TDS and calculation of a soil quality index based on the TDS, establishment of the MDS and calculation of a soil quality index based on the MDS, development of the soil quality prediction model based on machine learning, and generation of the soil quality evaluation dataset.

In the embodiment, in step S2, the establishing a TDS and a MDS, and calculating soil quality indices based on the TDS and the MDS includes collecting and processing the TDS, selecting a standard scoring function for an evaluation indicator of the TDS, selecting an evaluation indicator for the MDS, and calculating the soil quality indices.

In the embodiment, in step S2, the collecting and processing the TDS includes: selecting, based on a selection frequency of soil quality indicators and availability of indicator data, a soil physical indicator (bulk density), chemical indicators (organic matter, total nitrogen (TN), rapidly-available phosphorus, rapidly-available potassium, and pH), and biological indicators (microbial biomass carbon, microbial biomass nitrogen, sucrase, phosphatase, and urease) as the TDS for soil quality evaluation.

In the embodiment, in step S2, the selecting a standard scoring function for an evaluation indicator of the TDS includes: establishing the standard scoring function between the evaluation indicator and soil quality based on soil characteristics of different soil types and a correlation between the evaluation indicator and the soil quality.

In the embodiment, in step S2, the selecting an evaluation indicator for the MDS includes: calculating Norm values of the evaluation indicator as follows:

$${N}_{i k} = \sqrt{{\sum }_{i = 1}^{k} ( {U}_{ik}^{2} {λ}_{k} )}$$

where Nᵢₖ represents a comprehensive loading of an i-th indicator on top k principal components (PCs) with eigenvalues greater than 1; Uᵢₖ is a loading value of the i-th indicator on a k-th PC; and λₖ is an eigenvalue of the k-th PC.

In the embodiment, in step S2, the calculating soil quality indices includes: calculating a weight value of each indicator by using factor analysis. The soil quality indices are respectively calculated based on the TDS and the MDS by using following formula:

$$SQI = {\sum }_{i = 1}^{n} {W}_{i} \cdot {S}_{i}$$

where Wᵢ represents a weight of an i-th evaluation indicator, Sᵢ represents a membership degree of the i-th evaluation indicator, and n represents the number of participating evaluation indicators in each dataset.

In the embodiment, in step S3, the developing a soil quality prediction model based on machine learning includes: developing the soil quality prediction model and assessing accuracy of the soil quality prediction model, and developing a random forest regression (RFR) model.

In the embodiment, the developing the soil quality prediction model and assessing accuracy of the soil quality prediction model includes: quantifying performance of the prediction model with a coefficient of determination (R²), a root mean square error (RMSE), and a relative percent deviation (RPD) as follows:

$${R}^{2} = 1 - \frac{{\sum }_{i = 1}^{N} {( {y}_{i} - {\hat{y}}_{i} )}^{2}}{{\sum }_{i = 1}^{N} {( {y}_{i} - \overline{\hat{y}} )}^{2}}$$

$$RMSE = \sqrt{\frac{1}{N} {\sum }_{i = 1}^{N} {( {y}_{i} - {\hat{y}}_{i} )}^{2}}$$

$$RPD = \frac{S D}{RMSE}$$

$$SD = \sqrt{\frac{1}{N} {\sum }_{i = 1}^{N} {( {y}_{i} - \overline{y} )}^{2}}$$

where N is the number of samples; yᵢ and ŷᵢ represent a measured value and a corresponding predicted value, respectively; \hat{y̅}ᵢ represents an average value of predicted values; y̅ represents an average value of measured values. When RPD<1.4, predictive performance of the model is poor; when 1.4≤RPD<1.8, the model has a certain predictive ability and is capable of assessing the samples; when 1.8≤RPD<2.0, the model has a good predictive ability and is capable of quantitative prediction; when 2.0≤RPD<2.5, the model is capable of quantitative prediction with higher accuracy; when RPD≥2.5, the model is excellent and exhibits an extremely satisfactory quantitative prediction ability.

The present disclosure has the following beneficial effects:

1. By developing an ensemble learning prediction model, the present disclosure optimizes the process of verifying the TDS-based soil quality index via the MDS-based soil quality index in the prior art, thereby achieving the evaluation of soil quality under different organic material inputs. The overall framework includes four aspects: establishment of the TDS and calculation of the soil quality index based on the TDS, establishment of the MDS and calculation of the soil quality index based on the MDS, development of the soil quality prediction model based on machine learning, and generation of the soil quality evaluation dataset.

2. The present disclosure constructs MDS for farmland soil quality evaluation with reference to soil classification, and utilizes a decision tree regression (DTR) single model as well as random forest regression (RFR) and LightGBM ensemble models to predict the TDS-based soil quality index. An organic material-soil quality response prediction model based on the MDS is developed, and the response rules of soil quality to different organic material inputs under typical planting patterns is revealed, thereby providing a scientific basis and theoretical guidance for organic agriculture and ecological environmental protection.

### BRIEF DESCRIPTION OF THE DRAWINGS

To describe the technical solutions in the embodiments of the present disclosure or in the prior art more clearly, the accompanying drawings required for describing the embodiments or the prior art will be briefly described below. Apparently, the accompanying drawings in the following description show merely some embodiments of the present disclosure, and those of ordinary skill in the art may still derive other accompanying drawings from these accompanying drawings without creative efforts.

FIG. 1 is a diagram of an overall framework for soil quality evaluation based on a machine learning model according to the present disclosure.

### DETAILED DESCRIPTION OF THE EMBODIMENTS

The technical solutions in the embodiments of the present disclosure are described clearly and completely below with reference to the accompanying drawings in the embodiments of the present disclosure. Apparently, the described embodiments are merely some rather than all of the embodiments of the present disclosure. All other embodiments obtained by those of ordinary skill in the art based on the embodiments of the present disclosure without creative efforts should fall within the protection scope of the present disclosure.

### Embodiment 1

### 1. Overall Framework

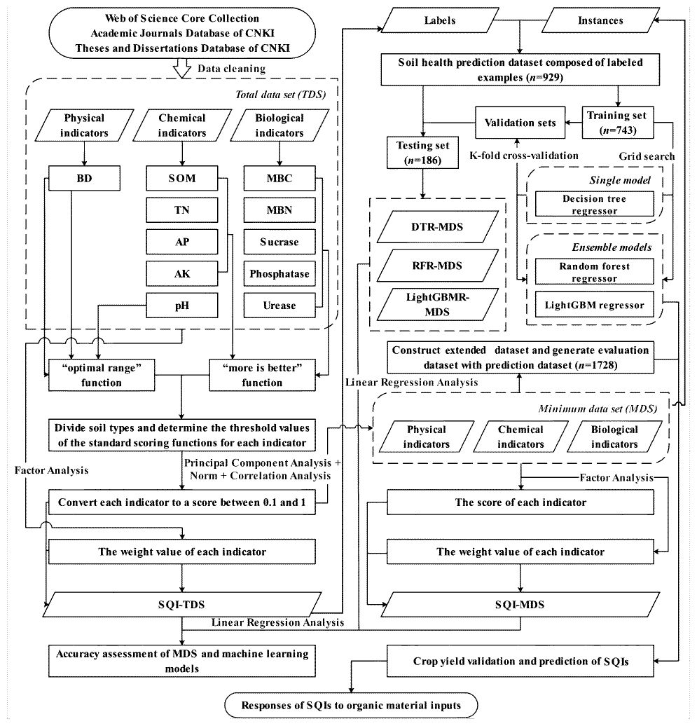

By developing an ensemble learning prediction model, the present disclosure optimizes the process of verifying a TDS-based soil quality index with an MDS-based soil quality index in the prior art, thereby achieving the evaluation of soil quality under different organic material inputs. The overall framework includes four aspects: establishment of a TDS and calculation of a soil quality index based on the TDS (as shown in the region 11 bounded by bold dashed lines in FIG. 1), establishment of the MDS and calculation of a soil quality index based on the MDS (as shown in the region 12 bounded by bold dashed lines in FIG. 1), development of a soil quality prediction model based on machine learning (as shown in the region 13 bounded by bold dashed lines in FIG. 1), and generation of a soil quality evaluation dataset (as shown in the region 14 bounded by bold dashed lines in FIG. 1). As supervised models are used in machine learning regression prediction problems, the key to this framework lies in the generation of “labels” and “instances.” The “labels” and “instances” also connect the development of machine learning prediction models with TDS and MDS based on a soil quality index method.

### 2. Establishing the TDS and the MDS, and Calculating Soil Quality Indices Based on the TDS and the MDS

### 2.1: Collecting and Processing the TDS

Soil quality is an intrinsic attribute of soil itself determined by seeking overall performance and a balance among different soil functions. This attribute cannot be directly obtained through sensory or instrumental analysis, but is inferred or quantified based on known external soil properties. In evaluation of soil quality, it is necessary to select soil quality indicators that most reflect an essence of the soil quality and show a relationship between various soil properties and soil functions. Therefore, selecting appropriate evaluation indicators is a prerequisite for obtaining a more realistic representation of actual soil quality.

In the present disclosure, based on a selection frequency of soil quality indicators and availability of indicator data, soil physical indicators (bulk density (BD)), chemical indicators (organic matter, total nitrogen, rapid-available phosphorus (AP), rapid-available potassium (AK), and pH), and biological indicators (microbial biomass carbon (MBC), microbial biomass nitrogen (MBN), sucrase, phosphatase, and urease) are selected as the TDS for soil quality evaluation.

The criteria for selecting qualified data in the present disclosure are as follows: (1) The research object is farmland soil. (2) All 11 preliminary selected indicators are included. (3) Each indicator is measured by using the same analytical method. (4) All processed data (including control data) are extracted. If there is no bulk density data for each processing, a background value of a sampling point is used as a uniform representation. (5) When the results are displayed in a numerical form, raw data is directly obtained from the tables or supplementary information in the paper. Otherwise, GetData Graph Digitizer (http://www.getdata-Graph-digitizer.com/index.php) is used for indirect retrieval. A total of 929 sets of sample data are collected, and each set of samples undergo data cleaning, including unit conversion and standardization, detection of outliers, etc., to form a soil quality prediction dataset. In addition, based on the Chinese Soil Database, the collected sampling points are divided into 18 soil types, including paddy soil, chestnut soil, fluvo-aquic soil, cinnamon soil, brown desert soil, loessial soil, dry red soil, heilu soil, calcareous soil, latosolic red soil, gray desert soil, alkaline soil, purple soil, aeolian sandy soil, yellow soil, chestnut soil, red soil, and red clay.

### 2.2: Selecting Standard Scoring Functions for Evaluation Indicators of the TDS

Standard scoring functions between the evaluation indicators and soil quality are established based on soil characteristics of different soil types and a correlation between the evaluation indicators and the soil quality. A standard scoring function is, in fact, a curve representing the relationship between the evaluation indicators and a crop growth effect curve. Thresholds for a standard scoring function are determined based on the suitability or restrictiveness of crop growth, and then the curve is converted into a broken line, and thus transforming the evaluation indicators into dimensionless values between 0.1 and 1 (i.e., indicator scores). Three types of standard scoring functions are generally used for continuous indicators: standard scoring function (SSF) 1, i.e., more is better (ceiling type); SSF2, i.e., optimal range (trapezoidal); SSF3, i.e., less is better (floor type). According to a long-term related research, for organic matter, total nitrogen, rapid-available phosphorus, rapid-available potassium, microbial biomass carbon, microbial biomass nitrogen, sucrase, phosphatase, and urease, the ceiling-type function can be employed to calculate membership values; while for bulk density and pH, the trapezoidal function is employed to calculate membership values (Table 1). For each indicator, after an appropriate standard scoring function is selected, it is necessary to determine thresholds such as the upper limit (U), lower limit (L), and optimal value (L). Finally, measured values of the soil quality indicators are substituted into the standard scoring function to calculate the scores.

A determination of the thresholds is crucial for the calculation of the standard scoring function. For bulk density, organic matter, rapid-available phosphorus, rapid-available potassium, and pH, the determination of the thresholds refer to the suggested schemes for the level classification of soil quality evaluation indicators for four major types of soils in China: paddy soil, red soil, fluvo-aquic soil, and black soil. For indicators without specific thresholds (total nitrogen, microbial biomass carbon, microbial biomass nitrogen, sucrase, phosphatase, and urease), since the ceiling-type function is employed for these indicators, the highest measured value is set to 1, the lowest measured value is set to 0.1, and other values are calculated by using the ceiling-type function at each sampling point (Liebig et al., 2001; Liu et al., 2015). In the case of soil classification, scores for each indicator are calculated separately for the 18 soil types (Table 1).

TABLE 1
Standard scoring functions for evaluation indicators of TDS

| Evaluation indicator | Function type | Standard scoring function |
|---|---|---|
| Organic matter (g*kg⁻¹) Total nitrogen (g*kg⁻¹) Rapid-available phosphorus (m*kg⁻¹) Rapid-available potassium | Ceiling-type function | $f ( X ) = \{ \begin{matrix}1. , X \geq U \\ 0.1 + 0.9 \times ( \frac{X - L}{U - L} ) , L < X < U \\ 0 \text{.1} , X \leq L\end{matrix}$ |
| (mg*kg⁻¹) | | |
| Microbial biomass carbon | | |
| (mg*kg⁻¹) | | |
| microbial biomass nitrogen | | |
| (mg*kg⁻¹) | | |
| Sucrase (mg C₆H₁₂O₆*g⁻¹*24h⁻¹) | | |
| Phosphatase (mg Phenol*g⁻¹*24h⁻¹) | | |
| Urease (mg NH₄⁺ − N*g⁻¹*24h⁻¹) | | |
| Bulk density (g*cm⁻³) | | |
| pH | Trapezoidal function | $f ( X ) = \{ \begin{matrix}0 \text{.1} , X \leq L , X \geq U \\ 0.1 + 0.9 \times ( \frac{X - L}{{O}_{1} - L} ) , L < X < {O}_{1} \\ 1. , {O}_{1} \leq X \leq {O}_{2} \\ 1. - 0. 9 \times ( \frac{X - {O}_{2}}{U - {O}_{2}} ) , {O}_{x} < X < U\end{matrix}$ |

Note: U represents the upper limit value of the function, L represents the lower limit value of the function, O₁ and O₂ represent optimal values of the function, and X represents a measured value.

### 2.3: Selecting Evaluation Indicators for the MDS

In the analysis of soil quality at a large spatial scale, direct application of TDS evaluation indicators incurs high data acquisition costs. The MDS achieves dimensionality reduction through principal component analysis, thereby reducing the analysis dimensions and effectively reflecting information of the TDS evaluation indicators.

Principal component analysis is performed on the preliminarily selected indicators, principal components (PCs) with eigenvalues greater than 1 are extracted. Indicators with absolute loading values greater than or equal to 0.5 on a same PC are divided into one group. If an indicator has absolute loading values greater than or equal to 0.5 on two PCs, the indicator is merged into a group with lower correlation with other indicators. If absolute loading values of an indicator on all PCs are less than 0.5, the indicator is assigned to a group with the highest absolute loading value. Norm values of indicators in each group are calculated, and in each group, indicators whose Norm values are within the 10% range of the maximum Norm value of the group are selected. The correlation between the selected indicators within each group is analyzed. If the correlation coefficient is greater than or equal to 0.5, the indicator with the highest Norm value is selected into the MDS. If the correlation coefficient is less than 0.5, both indicators are selected into the MDS. The Norm value represents a vector norm length of the indicator in a multidimensional space composed of components. A longer length indicates a larger comprehensive loading value across all PCs, implying a stronger ability to explain comprehensive information. A Norm value of an evaluation indicator is calculated as follows:

$${N}_{i k} = \sqrt{{\sum }_{i = 1}^{k} ( {U}_{ik}^{2} {λ}_{k} )} \qquad ( 1 )$$

where Nᵢₖ represents a comprehensive loading of an i-th indicator on top k principal components (PCs) with eigenvalues greater than 1; Uᵢₖ is a loading value of the i-th indicator on a k-th PC; and λₖ is an eigenvalue of the k-th PC.

### 2.4: Calculating Soil Quality Indices

A soil quality index integrates physical, chemical, and biological indicators of farmland soil. A higher soil quality index indicates better soil quality. Weight values represent the contribution of each evaluation indicator to soil quality. A larger weight value indicates greater importance of the indicator to the soil quality. To avoid interference from subjective factors, factor analysis method is used to calculate the weight values of each indicator. The soil quality indices are calculated based on the TDS and the MDS using the following formula:

$$SQI = {\sum }_{i = 1}^{n} {W}_{i} \cdot {S}_{i} \qquad ( 2 )$$

where Wᵢ represents a weight of an i-th evaluation indicator, Sᵢ represents a membership degree of the i-th evaluation indicator, and n represents the number of participating evaluation indicators in each dataset.

### 3. Developing a Soil Quality Prediction Model Based on Machine Learning

### 3.1: Developing the Prediction Model and Assessing Accuracy of the Prediction Model

The present disclosure employs a random forest regression (RFR) machine learning model to predict, on the basis of an MDS evaluation indicator system, a TDS-based soil quality index.

The development process of the machine learning prediction model involves three stages: data preparation, model training and validation, and model testing. In the present disclosure, the data preparation stage mainly includes constructing examples using the TDS and MDS to form a soil quality prediction dataset (where the examples are instances with labeled information), and splitting the prediction dataset (n=929) into a training set (n=743) and a testing set (n=186) in a ratio of 4:1. It should be noted that, a standard scoring function transforms the evaluation indicators into dimensionless values between 0.1 and 1, which is equivalent to a standardization process. During the model training and validation stage, optimal “hyperparameters” are selected through a grid search method (Table S2), and a 10-fold cross-validation method is employed on the training set to define a validation set. For RFR, the optimal hyperparameters are directly selected through grid search. During the model testing stage, the testing set data is fed into the trained model to obtain prediction results, which are then compared with traditional validation results based on the soil quality index. The coefficient of determination (R²), root mean square error (RMSE), and relative percent deviation (RPD) are used to quantify performance of the model:

$${R}^{2} = 1 - \frac{{\sum }_{i = 1}^{N} {( {y}_{i} - {\hat{y}}_{i} )}^{2}}{{\sum }_{i = 1}^{N} {( {y}_{i} - \overline{\hat{y}} )}^{2}} \qquad ( 3 )$$

$$RMSE = \sqrt{\frac{1}{N} {\sum }_{i = 1}^{N} {( {y}_{i} - {\hat{y}}_{i} )}^{2}} \qquad ( 4 )$$

$$RPD = \frac{S D}{RMSE} \qquad ( 5 )$$

$$SD = \sqrt{\frac{1}{N} {\sum }_{i = 1}^{N} {( {y}_{i} - \overline{y} )}^{2}} \qquad ( 6 )$$

where N represents the number of samples; yᵢ and ŷᵢ represent a measured value and a corresponding predicted value, respectively; \hat{y̅}ₗ represents an average value of predicted values; and y̅ represents an average value of measured values. Since complex interactions among soil components affect the distribution of specific soil properties, RPD values in soil science are significantly lower than that in most other fields. When RPD<1.4, predictive performance of the model is poor; when 1.4≤RPD<1.8, the model has a certain predictive ability and is capable of assessing samples; when 1.8≤RPD<2.0, the model has a good predictive ability and is capable of quantitative prediction; when 2.0≤RPD<2.5, the model is capable of quantitative prediction with higher accuracy; when RPD≥2.5, the model is excellent with an extremely satisfactory quantitative prediction ability.

### 3.2: Random Forest Regression (RFR) Model

RFR is a typical representative of the Bagging learning framework, and base learners (DTR) are constructed through randomness in samples and features, then a plurality of DTR models form a RFR model. Specifically, traditional DTR selects an optimal attribute for attribute partition from an attribute set of a current node (in the present disclosure, there are a total of 11 attributes), while in RFR, for each node of a base learner DTR, a subset containing k attributes is randomly selected from the attribute set of the node, and then an optimal attribute for partition is selected from this subset.

In the present disclosure, based on a soil quality training set, a sample is randomly selected and placed into a sampling set, and then the sample is returned to an initial training set, thereby allowing the sample to be potentially selected again in next sampling rounds. After m rounds of random sampling, a sampling set containing m samples is obtained, where some samples from the initial training set appear multiple times in the sampling set while others do not appear at all. Eventually, T sampling sets, each containing m training samples, are obtained, and a base learner (DTR) is trained based on each sampling set. Then, these base learners are combined. When combining predicted outputs, Bagging generally employs simple averaging for regression tasks.

### 4. Generation of a Soil Quality Extended Dataset and a Soil Quality Evaluation Dataset

The present disclosure primarily focuses on the variation characteristics of soil quality for three major crops, namely, rice, corn, and wheat, under different organic material inputs. Based on the MDS evaluation indicator system, relevant papers published before December 2022 are retrieved from the Web of Science Core Collection, as well as the Academic Journals Database of China National Knowledge Infrastructure (CNKI), China Doctoral Dissertations Full-text Database, and Chinese Master's Dissertations Full-Text Database. Data about soil quality indicators and crop yields under conditions of no fertilization and application of inorganic fertilizers (used as control treatments), as well as under various organic material inputs (experimental treatments) are extracted from these papers, thus forming the extended soil quality dataset. Additionally, relevant data from the soil quality prediction dataset are incorporated to jointly construct the soil quality evaluation dataset. Animal-source organic materials, include organic manure, farmyard manure, pig manure, cow manure, chicken manure, etc., while plant-source organic materials include straw, biochar, and green manure. The soil quality evaluation dataset includes a total of 1728 sets of sample data, covering 24 soil types.

### 5. Data Analysis Method

Principal component analysis and factor analysis are performed by using IBM SPSS Statistics 26. Model development is carried out in Python 3.9.7, with RFR utilizing the RandomForestRegressor class from the scikit-learn library. The creation of figures is implemented in R-4.1.3, with violin plots and box plots utilizing the ggstatsplot package.

The basic principles, main features, and advantages of the present disclosure are shown and described above. It should be understood by those skilled in the art that, the present disclosure is not limited by the above embodiments, and the above embodiments and the description only illustrate the principle of the present disclosure. Various changes and modifications may be made to the present disclosure without departing from the spirit and scope of the present disclosure, and such changes and modifications all fall within the claimed scope of the present disclosure.

What is claimed is:

1. An ensemble learning-based optimized evaluation method for an improvement effect of organic materials on soil quality, comprising following steps:
S1: formulating an overall framework;
S2: establishing a total dataset (TDS) and a minimum dataset (MDS), and calculating soil quality indices based on the TDS and the MDS;
S3: developing a soil quality prediction model based on machine learning;
S4: generating a soil quality extended dataset and a soil quality evaluation dataset; and
S5: analyzing data in the soil quality extended dataset and the soil quality evaluation dataset by using the soil quality prediction model, to evaluate improvement effect of organic materials on soil quality.

2. The ensemble learning-based optimized evaluation method according to claim 1, wherein in step S1, the overall framework comprises:
establishment of the TDS and calculation of a soil quality index based on the TDS, establishment of the MDS and calculation of a soil quality index based on the MDS, development of the soil quality prediction model based on the machine learning, and generation of the soil quality evaluation dataset.

3. The ensemble learning-based optimized evaluation method according to claim 1, wherein in step S2, the establishing a TDS and a MDS and calculating soil quality indices based on the TDS and the MDS comprises: collecting and processing the TDS, selecting a standard scoring function for an evaluation indicator of the TDS, selecting an evaluation indicator for the MDS, and calculating the soil quality indices.

4. The ensemble learning-based optimized evaluation method according to claim 3, wherein in step S2, the collecting and processing the TDS comprises: selecting, based on a selection frequency of soil quality indicators and availability of indicator data, a soil physical indicator of bulk density, chemical indicators of organic matter, total nitrogen, rapidly-available phosphorus, rapidly-available potassium, and pH, and biological indicators of microbial biomass carbon, microbial biomass nitrogen, sucrase, phosphatase, and urease as the TDS for soil quality evaluation.

5. The ensemble learning-based optimized evaluation method according to claim 3, wherein in step S2, the selecting a standard scoring function for an evaluation indicator of the TDS comprises: establishing the standard scoring function between the evaluation indicator and soil quality based on soil characteristics of different soil types and a correlation between the evaluation indicator and the soil quality.

6. The ensemble learning-based optimized evaluation method according to claim 3, wherein in step S2, the selecting an evaluation indicator for the MDS comprises: calculating Norm values of the evaluation indicator as follows:
$${N}_{i k} = \sqrt{{\sum }_{i = 1}^{k} ( {U}_{ik}^{2} {λ}_{k} )}$$
wherein Nᵢₖ represents a comprehensive loading of an i-th indicator on top k principal components (PCs) with eigenvalues greater than 1; Uᵢₖ is a loading value of the i-th indicator on a k-th PC; and λₖ is an eigenvalue of the k-th PC.

7. The ensemble learning-based optimized evaluation method according to claim 3, wherein in step S2, the calculating soil quality indices comprises: calculating a weight value of each indicator by using factor analysis; the soil quality indices are respectively calculated based on the TDS and the MDS by using following formula:
$$SQI = {\sum }_{i = 1}^{n} {W}_{i} \cdot {S}_{i}$$
wherein Wᵢ represents a weight of an i-th evaluation indicator, Sᵢ represents a membership degree of the i-th evaluation indicator, and n represents the number of participating evaluation indicators in each dataset.

8. The ensemble learning-based optimized evaluation method according to claim 1, wherein in step S3, the developing a soil quality prediction model based on machine learning comprises: developing the soil quality prediction model and assessing accuracy of the soil quality prediction model, and developing a random forest regression RFR model.

9. The ensemble learning-based optimized evaluation method according to claim 8, wherein the developing the soil quality prediction model and assessing accuracy of the soil quality prediction model comprising: quantifying performance of the prediction model with a coefficient of determination R², a root mean square error RMSE, and a relative percent deviation RPD as follows:
$${R}^{2} = 1 - \frac{{\sum }_{i = 1}^{N} {( {y}_{i} - {\hat{y}}_{i} )}^{2}}{{\sum }_{i = 1}^{N} {( {y}_{i} - \overline{\hat{y}} )}^{2}}$$
$$RMSE = \sqrt{\frac{1}{N} {\sum }_{i = 1}^{N} {( {y}_{i} - {\hat{y}}_{i} )}^{2}}$$
$$RPD = \frac{S D}{RMSE}$$
$$SD = \sqrt{\frac{1}{N} {\sum }_{i = 1}^{N} {( {y}_{i} - \overline{y} )}^{2}}$$
wherein N is the number of samples; yᵢ and ŷᵢ represent a measured value and a corresponding predicted value, respectively; \hat{y̅}ᵢ represents an average value of predicted values; y̅ represents an average value of measured values; when RPD<1.4, predictive performance of the soil quality prediction model is poor; when 1.4≤RPD<1.8, the model has a certain predictive ability and is capable of assessing the samples; when 1.8≤RPD<2.0, the soil quality prediction model has a good predictive ability and is capable of quantitative prediction; when 2.0≤RPD<2.5, the soil quality prediction model is capable of quantitative prediction with higher accuracy; when RPD≥2.5, the model is excellent and exhibits an extremely satisfactory quantitative prediction ability.
